(19) United States
(12) Patent Application Publication
(10) Pub. No.: US 2023/0255181 A1
(43) Pub. Date: Aug. 17, 2023

(54) AUTONOMOUS BIOMETRIC WEARABLE SYSTEM, ANIMAL MONITORING SYSTEM, ANXIETY TREATMENT SYSTEM FOR COMPANION ANIMALS AND METHODS OF USE

(71) Applicant: Erika Lobo, Overland Park, KS (US)

(72) Inventor: Erika Lobo, Overland Park, KS (US)

(21) Appl. No.: 18/107,033

(22) Filed: Feb. 8, 2023

Related U.S. Application Data
(60) Provisional application No. 63/311,108, filed on Feb. 17, 2022.

(51) Int. Cl. A01K 27/00; A01K 29/00; A61B 5/00; A61B 5/16

(52) U.S. Cl. CPC A01K 27/002; A01K 29/005; A61B 5/0004; A61B 5/165

(57) ABSTRACT

An autonomous biometric wearable system, animal monitoring system, animal monitoring system for companion animals and methods of use are presented. The biometric wearable for pets and animals includes a plurality of sensors and stimulators. The biometric wearable provides for detecting and notifying when stress and/or other anxiety-like symptoms are detected. The system then also provides for stimulation which may help in calming the animal or pet.

### CROSS-REFERENCE TO RELATED APPLICATIONS

The present application claims priority to the U.S. Provisional Patent Application No. 63/311,108 which was filed on Feb. 17, 2022, which is hereby incorporated by reference herein in its entirety, including any figures, tables, or drawings.

### FIELD OF THE DISCLOSURE

This disclosure relates to an autonomous biometric wearable system for companion animals. More specifically, and without limitation, this disclosure relates to an animal monitoring system. More specifically, and without limitation, this disclosure relates to an autonomous anxiety born symptom treatment system for companion animals and methods of using the same. More specifically, and without limitation, the present disclosure provides the state of the art with an autonomous biometric wearable that provides vital monitoring and treatment for anxiety born symptoms in companion animals. Furthermore, the present disclosure relates to a system for providing holistic feedback to owners on the wellbeing of companion animals.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright. Erika Lobo. All rights reserved.

### BACKGROUND OF THE DISCLOSURE

Companion animals are well known in the art. Companion animals such as dogs, cats, and the like regularly accommodate humans and/or are regularly left alone for long durations of time while owners are at work and the like. Companion animals generally don't have special training, but accompany humans and others for company, amusement, psychological support, and other reasons. Companion animals are not limited to dogs or cats, but also may be many other animals. Some companion animals may also be specially trained to assist humans with anxiety, stress, or other situations and/or symptoms.

While companion animals are commonly trained to assist human companions, many animals suffer from stress and/or anxiety, and/or other afflictions. In one example, dogs suffer regularly from anxiety. Like humans, companion animals anxiety and/or stress may be chronic and/or irregular and can be triggered by particular causes or triggers. This might be when a human and/or owner leaves a home for a long period of time for work, or the like. This may be caused by external environments and the like as well. Sometimes stress and/or anxiety or other situations and conditions can be overwhelming issues for companion animals.

Treatment of anxiety and/or stress in companion animals is complex. There may be a variety of stressors and factors causing stress and/or anxiety. Furthermore, there may be a variety of different types of treatment available for various circumstances. These treatments sometimes come too late or are never figured out at all. Sometimes various stimuli for stressors and/or anxiety are unpredictable or not seen at all. Medications or other treatments may be available, but represent a number of issues in treatment.

Complicating matters further is that anxiety and/or stress in companion animals may often be undetected. Because companion animals can not speak and generally can not communicate, they are unable to tell owners and/or humans about anxiety and/or stress they are experiencing.

Thus, there is a long-felt need in the art for a system for detecting and autonomously assisting in handling of stress and/or anxiety or similar in companion animals.

The disclosure herein provides these advantages and others as will become clear from the specification and claims provided.

### SUMMARY OF THE DISCLOSURE

An autonomous biometric wearable system for companion animals. More specifically, and without limitation, this disclosure relates to an animal monitoring system. More specifically, and without limitation, this disclosure relates to an autonomous anxiety born symptom treatment system for companion animals and methods of using the same. More specifically, and without limitation, the present disclosure provides the state of the art with an autonomous biometric wearable that provides vital monitoring and treatment for anxiety born symptoms in companion animals. Furthermore, the present disclosure relates to a system for providing holistic feedback to owners on the wellbeing of companion animals.

Said another way, the present disclosure provides the state of the art with an autonomous biometric wearable that provides vital monitoring and treatment for anxiety born symptoms (and other symptoms) in companion animals. Furthermore, the present disclosure provides the state of the art with feedback and other information to owners and/or a user on the wellbeing of companion animals.

Thus, it is a primary object of the disclosure to provide the state of the art with an autonomous biometric wearable system, animal monitoring system, animal monitoring system for companion animals and methods of use.

Another object of the disclosure is to provide an autonomous biometric wearable system, animal monitoring system, animal monitoring system for companion animals and methods of use that improves upon the understanding of conditions of companion animals.

Yet another object of the disclosure is to provide an autonomous biometric wearable system, animal monitoring system, animal monitoring system for companion animals and methods of use that improves upon the understanding of anxiety and anxiety levels of companion animals.

Another object of the disclosure is to provide an autonomous biometric wearable system, animal monitoring system, animal monitoring system for companion animals and methods of use that improves upon monitoring of conditions of companion animals.

Yet another object of the disclosure is to provide an autonomous biometric wearable system, animal monitoring system, animal monitoring system for companion animals and methods of use that improves upon monitoring of anxiety and other stressors of companion animals.

Another object of the disclosure is to provide an autonomous biometric wearable system, animal monitoring system, animal monitoring system for companion animals and methods of use that improves on treatment of conditions in companion animals.

Yet another object of the disclosure is to provide an autonomous biometric wearable system, animal monitoring system, animal monitoring system for companion animals and methods of use that improves on treatment of anxiety and stressors in companion animals.

Another object of the disclosure is to provide an autonomous biometric wearable system, animal monitoring system, animal monitoring system for companion animals and methods of use that provides monitoring of companion animals.

Yet another object of the disclosure is to provide an autonomous biometric wearable system, animal monitoring system, animal monitoring system for companion animals and methods of use that provides vital monitoring information related to companion animals.

Another object of the disclosure is to provide an autonomous biometric wearable system, animal monitoring system, animal monitoring system for companion animals and methods of use that provides ongoing data from biometric feedback for companion animals.

Yet another object of the disclosure is to provide an autonomous biometric wearable system, animal monitoring system, animal monitoring system for companion animals and methods of use that provides for health profile building of animal wellness.

Another object of the disclosure is to provide an autonomous biometric wearable system, animal monitoring system, animal monitoring system for companion animals and methods of use that provides for an easy understanding of companion animal wellbeing.

Yet another object of the disclosure is to provide an autonomous biometric wearable system, animal monitoring system, animal monitoring system for companion animals and methods of use that continuously monitor companion animal biometrics.

Another object of the disclosure is to provide an autonomous biometric wearable system, animal monitoring system, animal monitoring system for companion animals and methods of use that provides micro-electric stimulation.

Yet another object of the disclosure is to provide an autonomous biometric wearable system, animal monitoring system, animal monitoring system for companion animals and methods of use that provides micro-electric stimulation to the transcutaneous electrical nerve in companion animals.

Another object of the disclosure is to provide an autonomous biometric wearable system, animal monitoring system, animal monitoring system for companion animals and methods of use that provides biometric and visual feedback.

Yet another object of the disclosure is to provide an autonomous biometric wearable system, animal monitoring system, animal monitoring system for companion animals and methods of use that provides therapy for a companion animal.

Another object of the disclosure is to provide an autonomous biometric wearable system, animal monitoring system, animal monitoring system for companion animals and methods of use that informs as to a source of anxiety in a company animal.

Yet another object of the disclosure is to provide an autonomous biometric wearable system, animal monitoring system, animal monitoring system for companion animals and methods of use that includes a remote external camera.

Another object of the disclosure is to provide an autonomous biometric wearable system, animal monitoring system, animal monitoring system for companion animals and methods of use that provides for visual tracking and monitoring.

Yet another object of the disclosure is to provide an autonomous biometric wearable system, animal monitoring system, animal monitoring system for companion animals and methods of use that provides for location positioning.

Another object of the disclosure is to provide an autonomous biometric wearable system, animal monitoring system, animal monitoring system for companion animals and methods of use that provides an informative wearable for companion animals.

Yet another object of the disclosure is to provide an autonomous biometric wearable system, animal monitoring system, animal monitoring system for companion animals and methods of use that functions autonomously.

Another object of the disclosure is to provide an autonomous biometric wearable system, animal monitoring system, animal monitoring system for companion animals and methods of use that tracks historical data.

Yet another object of the disclosure is to provide an autonomous biometric wearable system, animal monitoring system, animal monitoring system for companion animals and methods of use that provides alerts.

Another object of the disclosure is to provide an autonomous biometric wearable system, animal monitoring system, animal monitoring system for companion animals and methods of use that can utilize global positioning systems.

Yet another object of the disclosure is to provide an autonomous biometric wearable system, animal monitoring system, animal monitoring system for companion animals and methods of use that are easy to use.

Another object of the disclosure is to provide an autonomous biometric wearable system, animal monitoring system, animal monitoring system for companion animals and methods of use that are safe to use.

Yet another object of the disclosure is to provide an autonomous biometric wearable system, animal monitoring system, animal monitoring system for companion animals and methods of use that are accurate.

Another object of the disclosure is to provide an autonomous biometric wearable system, animal monitoring system, animal monitoring system for companion animals and methods of use that can be used with various digital platforms.

Yet another object of the disclosure is to provide an autonomous biometric wearable system, animal monitoring system, animal monitoring system for companion animals and methods of use that provides a graphical user interface.

Another object of the disclosure is to provide an autonomous biometric wearable system, animal monitoring system, animal monitoring system for companion animals and methods of use that are quick and efficient.

Yet another object of the disclosure is to provide an autonomous biometric wearable system, animal monitoring system, animal monitoring system for companion animals and methods of use that are robust.

Another object of the disclosure is to provide an autonomous biometric wearable system, animal monitoring system, animal monitoring system for companion animals and methods of use that improves quality of life for a companion animal.

These and other objects, features, or advantages of the present disclosure will become apparent from the specification and claims.

### BRIEF DESCRIPTION OF THE DRAWINGS

The drawings accompanying and forming part of this specification are included to depict certain aspects of the disclosure.

FIG. 1 is a front view of the autonomous biometric wearable system, animal monitoring system, animal monitoring system for companion animals and methods of use; the view showing the front of the harness; the view showing harness straps; the view showing attachment features, attaching the straps to the harness; the view showing a battery location and a plurality of batteries; the view showing a leash latch point; the view showing status indicators; the view showing vital sign monitoring housing; the view showing a charge port.

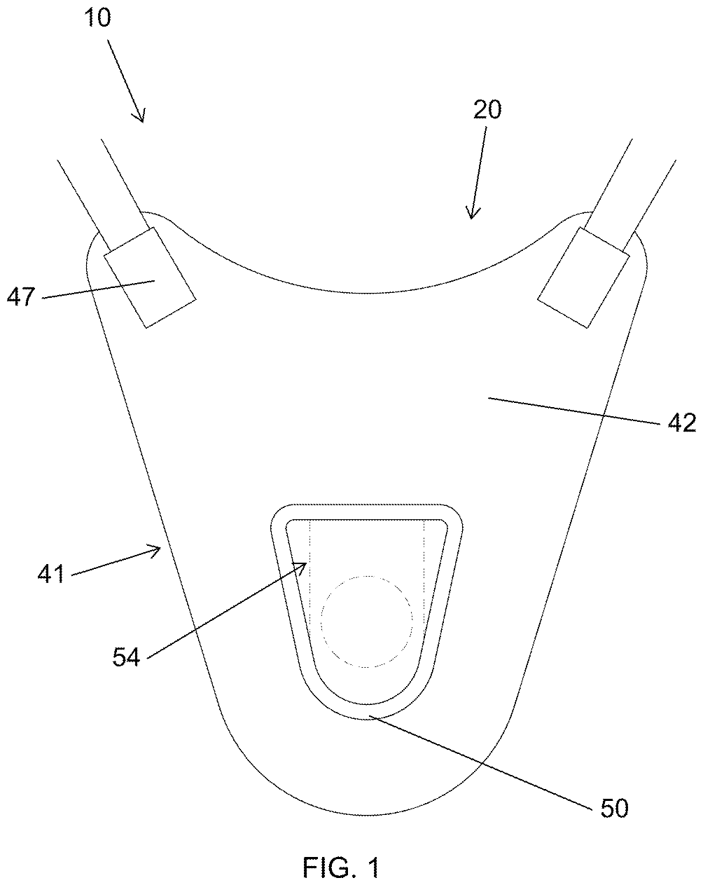

FIG. 2 is a rear view of the autonomous biometric wearable system, animal monitoring system, animal monitoring system for companion animals and methods of use; the view showing the back of the harness; the view showing harness straps; the view showing attachment features; the view showing a plurality of contact sensors; the view showing transcutaneous electrical nerve stimulation sensors; the view showing a plurality of contact sensors.

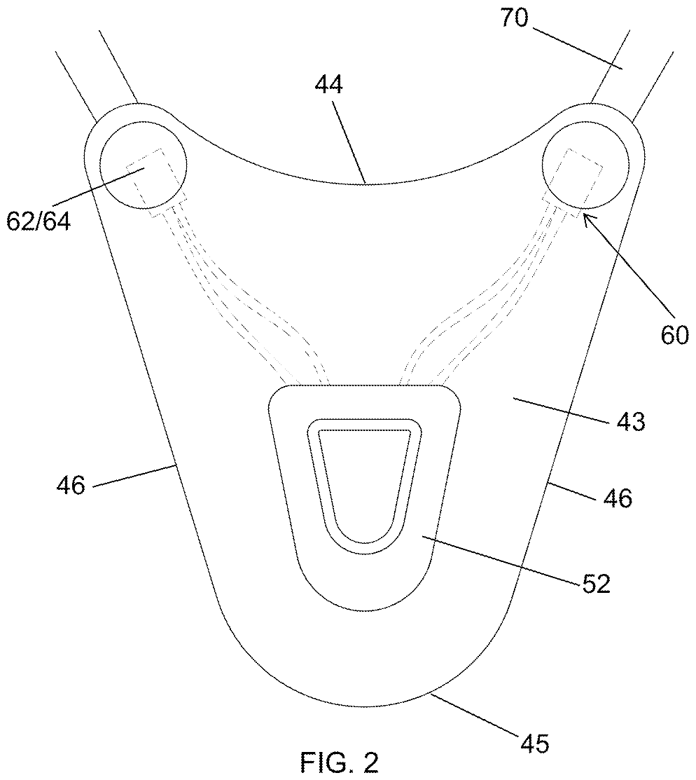

FIG. 3 is a top, perspective view of the autonomous biometric wearable system, animal monitoring system, animal monitoring system for companion animals and methods of use; the view showing the front of the harness; the view showing the fitting adjustment system; the view showing harness straps; the view showing attachment features, attaching the straps to the harness; the view showing a battery location and a plurality of batteries; the view showing a leash latch point; the view showing status indicators; the view showing vital sign monitoring housing; the view showing a charge port.

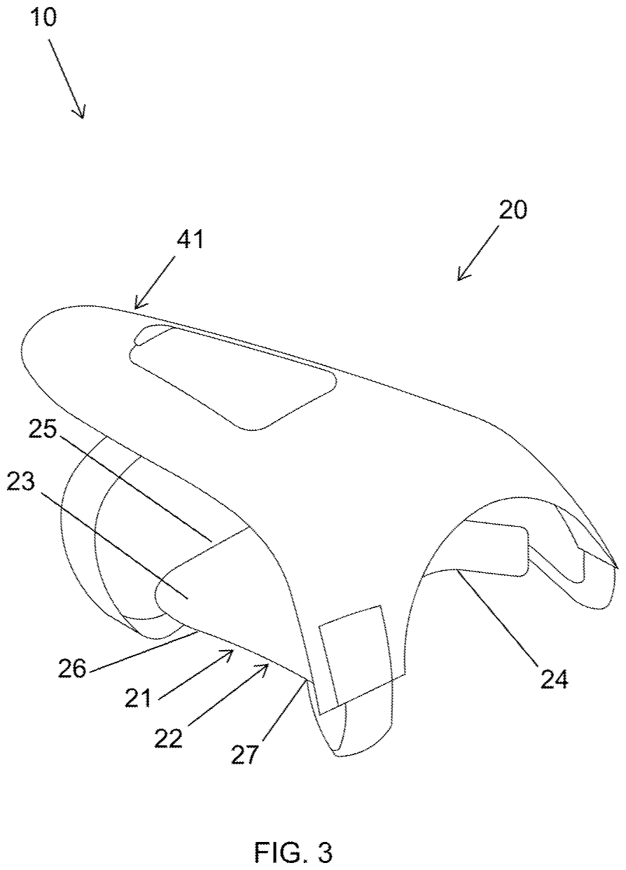

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer removable drive, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code, or virtual code, or framework code suitable for the disclosure herein, or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

System:

With reference to the figures, an autonomous biometric wearable system, animal monitoring system, animal monitoring system for companion animals and methods of use 10 are presented. Autonomous biometric wearable system, animal monitoring system, animal monitoring system for a companion animal and methods of use 10 (hereafter referred to as “companion animal monitoring system”, “monitoring system”, “anxiety monitoring and treatment system”, “anxiety system”, “biometric wearable”, or simply “system”) is formed of any suitable size, shape and design.

In the arrangement shown, as one example, irregular surface cleaning system 10 may comprise remote servers, databases, application servers, application databases, animal databases, mobile applications, and/or computers; all of which in continuity or as separate acts fulfill the functions disclosed herein. Companion animal monitoring system 10 also includes, in the embodiment(s) depicted, a graphical user interface, a user, an animal, a computing platform, a harness 20 having a front body 21 and a rear body 41, a plurality of harness straps 70, various adjustment feature, a plurality of wired connections, and a plurality of sensors, a computing system, an onboard computing system, a remote computer, a sensor system, an application server, a communication and/or control system, and a mobile computing application, among other components, features, and functions.

Harness:

In the arrangement shown, as one example, system 10 includes a harness 20 (also referred to as “body harness”, or simply collar”). Harness 20 is formed of any suitable size, shape, and design, and is configured to be worn by the companion animal and provide the various functionality of system 10, as disclosed herein. In the arrangement shown, as one example, the harness is formed of a wearable harness with multiple attachments. However, a collar or other wearable device is also hereby contemplated for use. Other examples of wearable devices include collars, ankle or leg attachment features, implanted devices, attached devices, and the like.

Front Body: In the arrangement shown, as one example, harness 20 includes a front body 21. Front body 21 is formed of any suitable size, shape, and design, and is configured to be secured to the chest and/or front body of a companion animal. In the arrangement shown, as one example, front body 21 includes an exterior surface 22, an interior surface 23, and extends a length between opposing sides 26 from a top 24 to a bottom 25. In the arrangement shown, as one example, front body 21 also includes a plurality of harness straps 27, a leash attachment feature, a housing, a plurality of batteries, a charging port, a plurality of status features, and a monitoring system, among other components, features and functionality.

Rear Body: In the arrangement shown, as one example, harness 20 includes a rear body 41. Rear body 41 is formed of any suitable size, shape, and design, and is configured to be secured to the back and/or rear body of a companion animal. In the arrangement shown, as one example, rear body 41 includes an exterior surface 42, an interior surface 43, and extends a length between opposing sides 46 from a top 44 to a bottom 45. In the arrangement shown, as one example, rear body 41 may also includes a plurality of harness straps 47, a leash attachment feature 50, a housing 52, a plurality of batteries 54, a charging port, a plurality of status features, and a monitoring system 60, a plurality of contact sensors 62, a transcutaneous electrical nerve stimulation sensor 64, and a plurality of wires, among other components, features and functionality.

In the arrangement shown, as one example, the harness system 20 may also include additional harness straps 70, various fitting adjustment features, various wired connections—including wiring to onboard computing components, and sensors—including environmental sensors and external camera features, among other features, components, and functionality.

Graphical User Interface:

In the arrangement shown, as one example, system 10 may include a graphical user interface. Graphical user interface is formed of any suitable size shape and design and is configured to allow a user to view interact with, manipulate, and visually access system data and information, information related thereto, and/or view various data for various environments and/or add information to system and/or environment and/or change the settings of the sensors and/or change the settings of operation.

Graphical user interface is an exemplary method by which systems of the present disclosure may operate and/or make programming changes to the operation of system 10. Employing graphical user interface, enhances a user's interactions with system 10 in the form of awareness and knowledge of data within the system 10. System 10 provides direct links to a user's history, settings, programming, and other information related to a particular function and/or a particular component and/or a particular user and/or a particular operation. In this way graphical user interface provides for a means for a user to make adjustments to the operation and functionality of system 10.

Display of Graphical User Interface: In the arrangement shown, as one example, system 10 includes a graphical user interface. Graphical user interface may include a display, which is configured to show and display information, including data, for review and interpretation by a user or plurality of users, or a plurality of users interacting with one another.

In the arrangement shown, as one example, an interactive user display is formed of a display screen, such as that of a desktop computer, laptop computer, monitor, tablet, smart phone, smart TV, projector, virtual reality display or any other device or form of a display. In the arrangement shown, as one example, interactive user display comprises a series of interactive user display pages, however, the interactive user display may consist of a single page or any other method of displaying information on a display as could be adapted to various size screens, devices, or user preferences. The interactive user display can display various information and/or functional information which is retrieved and/or requested.

User (or Plurality Thereof):

In the arrangement shown, as one example, system 10 includes a user. Users may be any user interacting with or utilizing the system 10. This may include viewing, controlling, analyzing, manipulating, and/or interacting with system 10. Users are not limited to a single user but may be a plurality of users. Generally speaking, a user might be an owner and/or medical provider monitoring and/or assisting in treatment of a companion animal.

Computing Platform:

In the arrangement shown, as one example, system 10 includes a computing platform (or “computer”, or “computer platform”). Computing platform is formed of any suitable size, shape, and design and is configured to provide computing support, power, and computing processing for both onboard computing functionality as well as communication for off-board or server computing functionality. In this way, an onboard computing system, among other components and features on top of the platform.

In the arrangement shown, as one example, system 10 includes a computer. Computer is formed of any suitable size, shape, and design and is configured to provide for the main off-board computing processing and implementation of computer handling of data from data gathering performed.

Onboard Computing System:

In one arrangement, as is shown, system 10 includes an onboard computing system (or “onboard computing device”). Onboarding computing system is formed of any suitable size, shape, and design and configured to handle onboard computing operations, as are necessary for the operation of system 10. Onboarding computing device is connected with electronic network and/or database and/or server or cloud via communication means, bluetooth communication, bluetooth low energy chip (BLE onboard), and may include a processor, a memory, a microcontroller, a printed circuit board, a microprocessor, a receiver/transceiver, may include at least one antenna, and a global positioning system, among other components.

Computing device may be formed of and/or include any computing device capable of displaying and manipulating data in the manners described herein. Computing device may include for example a desktop computer, a laptop computer, a tablet, smart phone, or any other computing device or other interactive device.

Computing device may be a single consolidated component, or alternatively, computing device may be formed of a plurality of interconnected components that may be co-located or located at different geographic locations. Computing device may be cloud based or it may be hardware based, or cloud capable. In addition, the connected components of computing device, including processor, memory, software and interactive user display, may be co-located with a computing device or located at different geographic locations, independent control features, and the like. That is, computing device may be made of any form of a device or system that individually or collectively performs the computing operations of system 10.

Printed Circuit Board: In the arrangement shown, as one example, system 10 may include a printed circuit board (“PCB”). PCB is formed of any suitable size, shape and design and is configured to facilitate carrying and/or holding other components and/or parts necessary to carry out various computation and/or related functions of system 10. PCB, as one example, might be a surface mounted PCB or a through-hole PCB. PCB, as one example, is green and facilitates connecting the components and/or parts of system 10 by the use of traces and or vias. Traces are formed of any suitable size, shape and design and are configured as lines electrically connecting the components and/or parts of system 10. Vias are formed of any suitable size, shape and design and are configured as holes that connect layers of traces together. Generally, as in shown, traces and vias are soldered to connect the components and/or parts to the PCB.

In an alternative embodiment, system 10 may not include an onboard computer device, but instead houses only a receiver and/or transceiver for sending and/or receiving information which is both sensed and/or information which is sensed and/or captured by the image capturing devices of system 10. In this way, system 10 may be connected to a mobile computing device via a cellular connection, a direct connection, or other connections which provide a means of receiving and/or sending communications.

Microprocessor: Microprocessor is any computing device that receives and processes information and outputs commands according to instructions stored in memory. Memory is any form of information storage such as flash memory, RAM memory, a hard drive, or any other form of memory. Memory may be included as a part of or operably connected to a microprocessor. A receiver/transceiver is connected to a microprocessor. A receiver is used if one way communication is utilized, whereas a transceiver is used if two-way communication is utilized (hereinafter “transceiver”). Receiver/transceiver is connected with an antenna, such as a monopole antenna, a loop antenna, a fractal antenna, or any other form of an antenna. Antenna receives wireless signals from any other device, transmits these signals to receiver/transceiver which processes these signals and then transmits these processed signals to microprocessor which processes these signals according to instructions stored in memory. In one arrangement, system 10 re-transmits operating commands signals through receiver/transceiver so as to similarly control over-the-air communication. Communication is any form of wireless signals, or wired signals, through the air, such as a conventional remote signal, a cell phone, a wireless device, an internet connected device, a hard-wired device, or any other device capable of transmitting remote control signals.

Memory: In the arrangement shown, as one example, system 10 includes a memory. Memory may be formed of any suitable size, shape and design and is configured to facilitate selective storage and retrieval of data (including data) in association with computing devices, processors, software and interactive user display. Memory may be a single component, such as a single chip or drive or other memory device, or alternatively memory may be formed of a plurality of memory or storage components that are connected to one another that may be co-located or located at different geographic locations.

Remote Computing System:

In one arrangement, as is shown, system 10 includes a remote computing system (or “remote computing device”). Remote computing device is formed of any suitable size, shape, and design and configured to handle onboard computing operations, as are necessary for the operation of system 10. Remote computing device is connected with electronic network and/or database and/or server or cloud via communication means and includes a processor, a memory, a microcontroller, a printed circuit board, a microprocessor, a receiver/transceiver, may include at least one antenna, a power supply, and a communications system, among other components.

Computing devices may be formed of any computing device capable of displaying and manipulating data in the manners described herein. Computing devices may include for example a desktop computer, a laptop computer, a tablet, smart phone, or any other computing device or other interactive device.

Computing devices may be a single consolidated component, or alternatively, computing devices may be formed of a plurality of interconnected components that may be co-located or located at different geographic locations. Computing devices may be cloud based or it may be hardware based, or cloud capable. In addition, the connected components of computing devices, including processor, memory, software and interactive user display, may be co-located with computing devices or located at different geographic locations. That is, computing devices may be made of any form of a device or system that individually or collectively performs the computing operations of system 10.

Printed Circuit Board: In the arrangement shown, as one example, system 10 includes a printed circuit board (“PCB”). PCB is formed of any suitable size, shape and design and is configured to facilitate carrying and/or holding other components and/or parts necessary to carry out various computation and/or related functions of system 10. PCB, as one example, might be a surface mounted PCB or a through-hole PCB. PCB, as one example, is green and facilitates connecting the components and/or parts of system 10 by the use of traces and or vias. Traces are formed of any suitable size, shape and design and are configured as lines electrically connecting the components and/or parts of system 10. Vias are formed of any suitable size, shape and design and are configured as holes that connect layers of traces together. Generally, as in shown, traces and vias are soldered to connect the components and/or parts to the PCB.

In an alternative embodiment, system 10 may not include an onboard computer device, but instead houses only a receiver and/or transceiver for sending and/or receiving information which is both sensed and/or information which is sensed and/or captured by the image capturing devices of system 10. In this way, system 10 may be connected to a mobile computing device via a cellular connection, a direct connection, or other connections which provide a means of receiving and/or sending communications.

Microprocessor: Microprocessor is any computing device that receives and processes information and outputs commands according to instructions stored in memory. Memory is any form of information storage such as flash memory, RAM memory, a hard drive, or any other form of memory. Memory may be included as a part of or operably connected to a microprocessor. A receiver/transceiver is connected to a microprocessor. A receiver is used if one way communication is utilized, whereas a transceiver is used if two-way communication is utilized (hereinafter “transceiver”). Receiver/transceiver is connected with an antenna, such as a monopole antenna, a loop antenna, a fractal antenna, or any other form of an antenna. Antenna receives wireless signals from any other device, transmits these signals to receiver/transceiver which processes these signals and then transmits these processed signals to microprocessor which processes these signals according to instructions stored in memory. In one arrangement, system 10 re-transmits operating commands signals through receiver/transceiver so as to similarly control over-the-air communication. Communication is any form of wireless signals, or wired signals, through the air, such as a conventional remote signal, a cell phone, a wireless device, an internet connected device, a hard-wired device, or any other device capable of transmitting remote control signals.

Memory: In the arrangement shown, as one example, system 10 includes a memory. Memory may be formed of any suitable size, shape and design and is configured to facilitate selective storage and retrieval of data (including data) in association with computing devices, processors, software and interactive user display. Memory may be a single component, such as a single chip or drive or other memory device, or alternatively memory may be formed of a plurality of memory or storage components that are connected to one another that may be co-located or located at different geographic locations.

Sensor System:

In the arrangement shown, as one example, system 10 includes a sensor system. Sensor system is formed of any suitable size, shape, and design and may include one or more sensors and/or one or more sensing technologies. In the arrangement shown, as one example, sensor system is configured to detect and communicate information related to system 10 as well as the surroundings and/or environment of system 10.

In the arrangement shown, as one example, various sensors are utilized within system 10 to detect system status such as distance, temperature changes, and other operating status parameters within system 10.

In the arrangement shown, as one example, a sensory system and perhaps a LIDAR sensor system is utilized to understand the environment surrounding system 10 is engaging with. This might include both distance and imaging sensors and/or camera sensors.

Other Sensors:

In addition to the distance and image sensors discussed herein, system 10 may also include other sensors, such as temperature sensors, moisture sensors, heat sensors, light sensors, motion sensors, and other sensors. In the arrangement shown, as one example, system 10 includes at least one other sensor. Other sensors are formed of any suitable size, shape and design and are configured to facilitate sensing of surfaces and/or environmental information and converting the characteristics of the outside space and/or environment into computer readable information. Other sensors are used to detect and respond to some type of input from the physical environment.

Other sensors may be used for sensing a single component of an environment. For example, the specific input of other sensors may be light, heat, motion, moisture, pressure, or any one of a great number of other information related to system 10. Another sensor is a device, module, or subsystem whose purpose is to detect events or changes in system 10 and send the information to other electronics, frequently a computer processor. The output of another sensor is generally a signal that is generally converted to human-readable display at the sensor location or transmitted electronically over a network for reading or further processing. Both analog sensors and/or digital sensors are hereby contemplated for use. In one arrangement, another sensor and/or microsensor sends information to a processor for use with other electronics.

Application Server:

In the arrangement shown, as one example, system 10 may comprise remote servers, databases, and/or computers that fulfill the functions disclosed and described herein. In the embodiment depicted, system 10 comprises an application server. Application server comprises one or more computer systems adapted to transmit and receive data regarding selected datasets related to various users and/or datasets related to multiple users. Application server is adapted to query databases, and may utilize unique identification codes, to retrieve information and associated information related to system 10.

Application server may transmit data and/or related documents with respect to a system 10. Application server is also adapted to query a database. Additionally, application server may communicate with a cloud computing system or a mobile application, which can also be adapted to present the data in a form conducive to being viewed on a mobile device and/or handheld device.

As one of ordinary skills in the art may understand, application server, database, and other databases mentioned herein may be implemented in one or more servers. Furthermore, each may be on multiple servers to increase system efficiency, especially when handling large data gathering, following extended guidelines, extended ranges, ranges discussed herein. Additionally, multiple servers may have mirrored data to prevent data loss in case of disk failure and/or to decrease access and response times for database queries. In alternative embodiments, application server, and other database procedures may be carried out on computer-readable instructions and data stored on the customer's mobile computing device.

In addition to the above identified features, options, controls, and components, system 10 may also include other features and functionalities, among other options, controls, and components.

In Operation:

Various examples of operations and methods of use are hereby contemplated.

It will be appreciated by those skilled in the art that other various modifications could be made to the system, process, and method of use without parting from the spirit and scope of this disclosure. All such modifications and changes fall within the scope of the claims and are intended to be covered thereby.

What is claimed:

1. A companion animal monitoring system, comprising:
a harness;
the harness having a front body and a rear body;
the front body of the harness having an exterior surface and an interior surface;
the front body of the harness having a top and a bottom and opposing sides;
the rear body of the harness having an exterior surface and an interior surface;
the rear body of the harness having a top and a bottom and opposing sides;
the harness having a housing;
the harness having a charging port;
the harness having a plurality of status indicators;
the harness having a monitoring system;
the harness having a plurality of contact sensors;
the harness having a plurality of transcutaneous electrical nerve stimulation sensors.

2. The system of claim 1, further comprising:
the harness having a plurality of strap attachments.

3. The system of claim 1, further comprising:
the harness having a plurality of strap attachments;
a plurality of harness straps;
the plurality of straps each having fitting adjustments;
the plurality of straps each having wired connections;
the plurality of straps each having a plurality of sensors;
the plurality of straps having a camera feature.

4. The system of claim 1, further comprising:
the harness formed in the shape of a collar;
wherein the harness is configured to fit around the neck of a pet.

5. The system of claim 1, further comprising:
the harness formed in the shape of a body harness;
the body harness configured to fit on the back and chest of a pet.

6. The system of claim 1, further comprising:
the harness having a plurality of batteries.

7. The system of claim 1, further comprising:
the harness having a plurality of rechargeable batteries.

8. The system of claim 1, further comprising:
the harness having a plurality of wires.

9. The system of claim 1, further comprising:
wherein the charging port is an induction charging port.

10. The system of claim 1, further comprising:
wherein the charging port is an induction charging port;
wherein the induction charging port is configured to secure a battery pack for transfer of a charge.

11. The system of claim 1, further comprising:
a computing platform;
a computing system;
an onboard computing system;
a remote computing system;
a sensor system;
an application server;
a communications system;
a mobile computing application;
a graphical user interface.

12. The system of claim 1, further comprising:
the harness having a leash attachment.

13. The system of claim 1, further comprising:
wherein the monitoring system utilizes the contact sensors to detect vitals of a pet.

14. The system of claim 1, further comprising:
wherein the monitoring system utilizes the contact sensors to detect vitals of a pet;
the vitals of a pet including heart rate, step count, breathing rate.

15. The system of claim 1, further comprising:
wherein the monitoring system utilizes the contact sensors to detect vitals of a pet;
wherein the plurality of transcutaneous electrical nerve stimulation sensors are configured to stimulate a pet so as to relax the pet, reducing anxiety and stress;
wherein the plurality of transcutaneous electrical nerve stimulation sensors receive a processing signal for a set of various stimulations based on a predetermined set of rules;
wherein the predetermined set of rules interprets the vitals of a pet to determine what set of various stimulations to activate;
wherein activation is the triggering of one a stimulation routine selected from the set of various stimulations.

16. A companion animal monitoring and treatment system, the system comprising:
a harness;
the harness extending a length from a first end to a second end;
the harness having a charging port;
the harness having a monitoring system;
the harness having a plurality of transcutaneous electrical nerve stimulation sensors;
wherein the monitoring system utilizes the contact sensors to detect vitals of a pet;
wherein the plurality of transcutaneous electrical nerve stimulation sensors are configured to stimulate a pet so as to relax the pet, reducing anxiety and stress;
wherein the plurality of transcutaneous electrical nerve stimulation sensors receive a processing signal for a set of various stimulations based on a predetermined set of rules;
wherein the predetermined set of rules interprets the vitals of a pet to determine what set of various stimulations to activate;
wherein activation is the triggering of one a stimulation routine selected from the set of various stimulations.

17. The system of claim 16, further comprising:
an onboard computing system;
wherein processing of vitals and triggering of stimulations is determined onboard a harness processing system of the onboard computing system.

18. The system of claim 16, further comprising:
a remote computing system;
wherein processing of vitals and triggering of stimulations is determined remotely;
a communication system;
wherein the harness communicates through a transceiver.

19. The system of claim 16, further comprising:
wherein the monitoring system utilizes the contact sensors to detect vitals of a pet.

20. A method of autonomously monitoring biometric information for a companion animal and
treating anxiety for a companion animal, comprising the steps:
attaching a monitoring system to an animal;
detecting vitals of the animal;
interpreting vitals of the animal;
determining a set of stimulations to sooth the animal;
triggering one of the set of stimulations to sooth the animal;
soothing the animal through stimulations of a transcutaneous electrical nerve stimulation.
